(19) United States
(12) Patent Application Publication
(10) Pub. No.: US 2024/0299250 A1
(43) Pub. Date: Sep. 12, 2024

(54) Nasal Spray Reconstitution System

(71) Applicant: BECTON, DICKINSON AND COMPANY, Franklin Lakes, NJ (US)

(72) Inventors: Longxiang Huang, Suzhou (CN); Huasheng Huang, Suzhou (CN); Jian Ma, Suzhou (CN); Yan Liu, Shanghai (CN)

(21) Appl. No.: 18/275,288

(22) PCT Filed: Feb. 14, 2022
(86) PCT No.: PCT/US22/16275

§ 371 Date: Aug. 1, 2023

(30) Foreign Application Priority Data
Feb. 14, 2021 (CN) 202110186024.0

(51) Int. Cl. A61J 1/20; A61J 1/06

(52) U.S. Cl. CPC A61J 1/2096; A61J 1/065; A61J 1/201; A61J 1/202; A61J 1/2055; A61J 1/2086

(57) ABSTRACT

A nasal spray reconstitution system (10) includes a vial adapter (12) having a body, a vial connector (46) configured to secure the body to a vial, a syringe connector (48) configured to secure the body to a syringe barrel, and a transfer spike (50). The system further includes a withdrawal member (14) defining a withdrawal opening and a central passageway, with the withdrawal member secured to the transfer spike via a spike connector (74), and a nozzle spray attachment (16) having a syringe attachment configured to be secured to a syringe barrel and a nozzle opening, with the nozzle spray attachment defining a central passageway in fluid communication with the syringe attachment and the nozzle opening.

### CROSS-REFERENCE TO RELATED APPLICATION

The present application claims priority to Chinese Application No. 202110186024.0, filed Feb. 14, 2021, entitled “Nasal Spray Reconstitution System”, the entire disclosure of which is hereby incorporated by reference in its entirety.

### BACKGROUND OF THE INVENTION

### Field of the Invention

The present invention relates to a nasal spray reconstitution system.

### Description of Related Art

Nasal spray delivery of medicament, such as a respiratory vaccine, is known from the market, but is growing in popularity due to a variety of factors, such as patient acceptance, safety, and immune response. The nasal spray delivery may be provided using a nozzle spray connector attached to a syringe containing the medicament. In a general manner, the medicament may be lyophilized and require reconstitution prior to delivery, because the medicament needs to be stored in specific conditions. Lyophilized medicament, such as a lyophilized vaccine, may be more stable than a pre-filled liquid drug. Reconstitution is typically accomplished by adding a diluent to a vial containing the lyophilized medicament and shaking the vial to fully dissolve the lyophilized medicament. Reconstitution devices are available to facilitate the reconstitution and transfer of the medicament. However, reconstitution of lyophilized medicament is not known regarding nasal spray delivery, and there exists a need for providing such a nasal spray delivery allowing the reconstitution of the lyophilized drug before delivery.

### SUMMARY OF THE INVENTION

In one aspect or embodiment, a nasal spray reconstitution system includes a vial adapter with a body having a first end and a second end positioned opposite the first end, a vial connector configured to secure the body to a vial, a syringe connector configured to secure the body to a syringe barrel, and a transfer spike having a pointed end and defining a transfer opening, where the body, the syringe connector, and the transfer spike define a passageway in fluid communication with the transfer opening of the transfer spike. The system further includes a withdrawal member having a first end and a second end positioned opposite the first end, with the withdrawal member defining a withdrawal opening and a central passageway, where the withdrawal member is secured to the transfer spike via a spike connector. The withdrawal opening of the withdrawal member is in fluid communication with the passageway of the vial adapter. The system also includes a nozzle spray attachment having a first end and a second end positioned opposite the first end, with the nozzle spray attachment having a syringe attachment configured to be secured to a syringe barrel and a nozzle opening. The nozzle spray attachment defines a central passageway in fluid communication with the syringe attachment and the nozzle opening.

The system may include a syringe having a syringe barrel and a plunger rod, with the syringe connector of the vial adapter secured to the syringe barrel. The system may include a vial containing a lyophilized medicament. The system may include an ampoule containing a diluent.

The system may include a dose divider having a plunger rod attachment portion configured to attach the dose divider to a plunger rod of a syringe. The dose divider may include first and second grip portions and C-shaped body, where movement of the grip portions toward each other increase an inner diameter of the C-shaped body.

The syringe connector may be a luer connection. The spike connector may be a luer connection. The withdrawal member may taper in width from the first end of the withdrawal member to the second end of the withdrawal member. A filter may be received within the central passageway of the withdrawal member. The body of the vial adapter may include a grip surface configured to be grasped by a hand of a healthcare provider.

The transfer opening of the transfer spike may include a first opening and a second opening positioned opposite the first opening. The first and second openings of the transfer spike may each have a length defined by a direction extending from the first end of the body to the second end of the body and a width defined by a direction extending perpendicular to the length of the first and second openings, where the length of the first and second openings is larger than the width of the first and second openings. The length of the first and second openings is at least 5 times larger than the width of the first and second openings.

In a further aspect or embodiment, a method of using the nasal spray reconstitution includes: inserting the withdrawal member into a container; withdrawing diluent from the container into a syringe connected to the vial adapter; disconnecting the withdrawal member from the vial adapter; attaching the vial adapter to a vial comprising a lyophilized medicament; transferring the diluent to the vial using the syringe to reconstitute the lyophilized medicament; withdrawing the reconstituted medicament into the syringe; removing the vial adapter from the syringe; and attaching the nozzle spray attachment to the syringe. The method may further include attaching a dose divider to a plunger rod of the syringe. The container may be a glass ampoule.

### BRIEF DESCRIPTION OF THE DRAWINGS

FIG. 1 is a perspective view of a nasal spray reconstitution system according to one aspect or embodiment of the present application.

FIG. 2 is a perspective view of a vial adapter of the system of FIG. 1 according to one aspect or embodiment of the present application.

FIG. 3 is a front view of the vial adapter of FIG. 1.

FIG. 4 is a cross-sectional view taken along line 4-4 in FIG. 3.

FIG. 5 is a front view of the vial adapter of FIG. 1, showing the vial adapter connected to a withdrawal member.

FIG. 6 is a cross-sectional view taken along line 6-6 in FIG. 5.

FIG. 7 is a perspective view of a dose divider of the system of FIG. 1 according to one aspect or embodiment of the present application.

FIG. 8 is a top view of the dose divider of FIG. 7.

FIG. 9 is a perspective view of a nozzle spray attachment of the system of FIG. 1 according to one aspect or embodiment of the present application.

FIG. 10 is a front view of the nozzle spray attachment of FIG. 9.

FIG. 11 is a cross-sectional view taken along line 11-11 in FIG. 10.

FIG. 12 is a schematic view of a method of using a nasal spray reconstitution system according to one aspect or embodiment of the present application.

### DETAILED DESCRIPTION

For purposes of the description hereinafter, the terms such as “end”, “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting. Further, it is to be understood that the invention may assume various alternative variations and step sequences, except where expressly specified to the contrary.

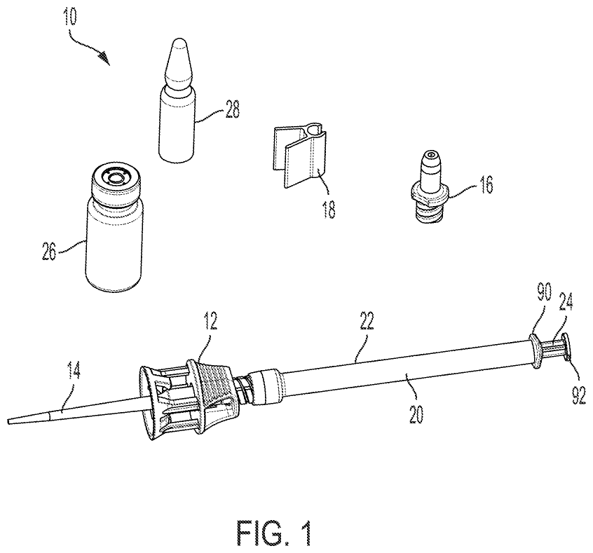

Referring to FIG. 1, a nasal spray reconstitution system 10 according to one aspect or embodiment includes a vial adapter 12, a withdrawal member 14, and a nozzle spray attachment 16. In one aspect or embodiment, the system 10 further includes a dose divider 18. In one aspect or embodiment, the system 10 includes a syringe 20 having a syringe barrel 22 and a plunger rod 24. In a further aspect or embodiment, the system 10 includes a vial 26 containing a lyophilized medicament, such as a lyophilized vaccine, and/or an ampoule 28 containing a diluent. As detailed below, the nasal spray reconstitution system 10 is configured to reconstitute a lyophilized medicament and deliver the medicament nasally via the nozzle spray attachment 16.

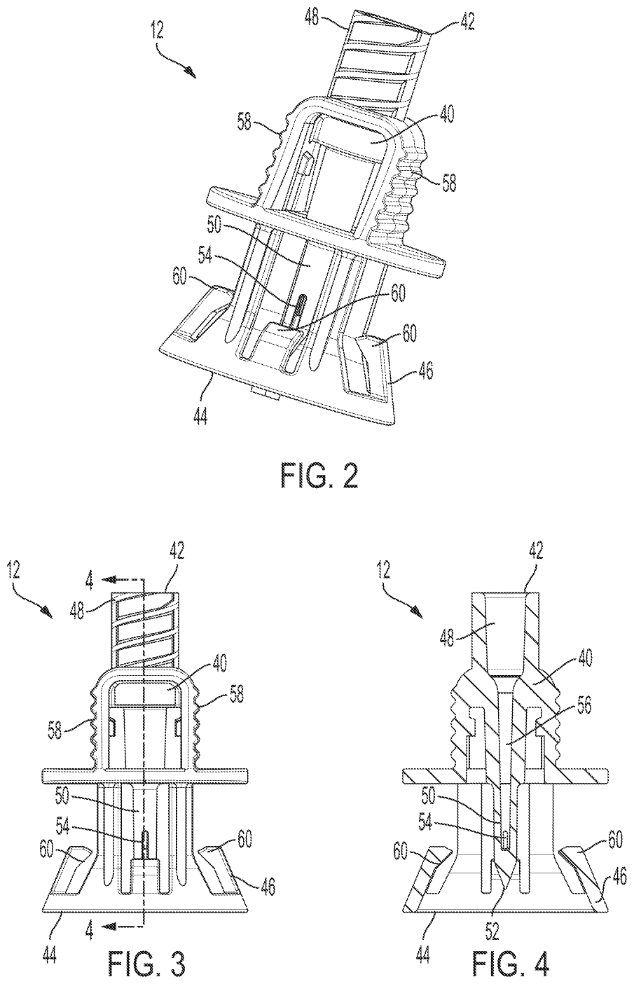

Referring to FIGS. 2-4, the vial adapter 12 includes a body 40 having a first end 42 and a second end 44 positioned opposition the first end 42, a vial connector 46 configured to secure the body 40 to the vial 26, a syringe connector 48 configured to secure the body 40 to the syringe barrel 22, and a transfer spike 50 having a pointed end 52 and defining a transfer opening 54. The body 40, the syringe connector 48, and the transfer spike 50 define a passageway 56 in fluid communication with the transfer opening 54 of the transfer spike 50. The body 40 of the vial adapter 12 includes a grip surface 58 configured to be grasped by a hand of a healthcare provider. The grip surface 58 includes two separate portions positioned opposite from each other, although other suitable arrangements may be utilized. The syringe connector 48 is a luer connector, although other suitable connection arrangements may be utilized. The vial connector 46 includes a plurality of projections 60. The plurality of projections 60 are configured to deflect radially outward as the vial adapter 12 is pressed onto the vial 26 and return to their original position such that the plurality of projections 60 are positioned underneath a cap of the vial 26 to secure the vial adapter 12 to the vial 26. In one aspect or embodiment, the vial adapter 12 may be similar to the vial adapter shown and described in International Application Publication No. WO 2019/219383.

The transfer opening 54 of the transfer spike 50 includes a first opening and a second opening positioned opposite the first opening, although other suitable arrangements and one or more openings may be utilized. The first and second openings 54 of the transfer spike 50 each have a length defined by a direction extending from the first end 42 of the body 40 to the second end 44 of the body 40 and a width defined by a direction extending perpendicular to the length of the first and second openings 54. The length of the first and second openings 54 is larger than the width of the first and second openings 54. The first and second openings 54 are configured to remove all of the fluid from the vial 26 when the vial adapter 12 is secured to the vial 26 via the vial connector 46. In further aspects or embodiments, the first and second openings 54 are configured to remove 95% or more, 96% or more, 97% or more, 98% or more, or 99% or more of the fluid from the vial 26 when the vial adapter 12 is secured to the vial 26 via the vial connector 46. In one aspect or embodiment, the length of the first and second openings 54 is at least 5 times larger than the width of the first and second openings 54.

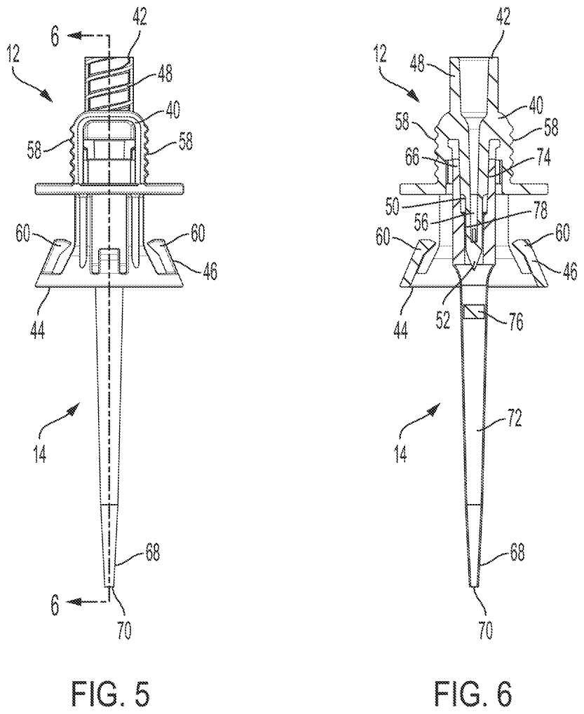

Referring to FIGS. 5 and 6, the withdrawal member 14 has a first end 66 and a second end 68 positioned opposite the first end 66, with the withdrawal member 14 defining a withdrawal opening 70 and a central passageway 72. The withdrawal member 14 is secured to the transfer spike 50 via a spike connector 74, with the withdrawal opening 70 of the withdrawal member 14 in fluid communication with the passageway 56 of the vial adapter 12. The spike connector 74 is a luer connector, although other suitable connection arrangements may be utilized. The spike connector 74 provides a liquid-tight connection between the withdrawal member 14 and the vial adapter 12. The withdrawal member 14 tapers in width from the first end 66 of the withdrawal member 14 to the second end 68 of the withdrawal member 14. In one aspect or embodiment, a filter 76 is received within the central passageway 72 of the withdrawal member 14. The filter 76 is configured to filter out any particulates when transferring fluid through the withdrawal member 14. As shown in FIG. 6, the transfer spike 50 of the vial adapter 12 and the withdrawal member 14 define an annulus 78 to allow the fluid communication through the central passageway 72 of the withdrawal member 14, through the transfer opening 54 of the transfer spike 50, and into the passageway 56 of the vial adapter 12.

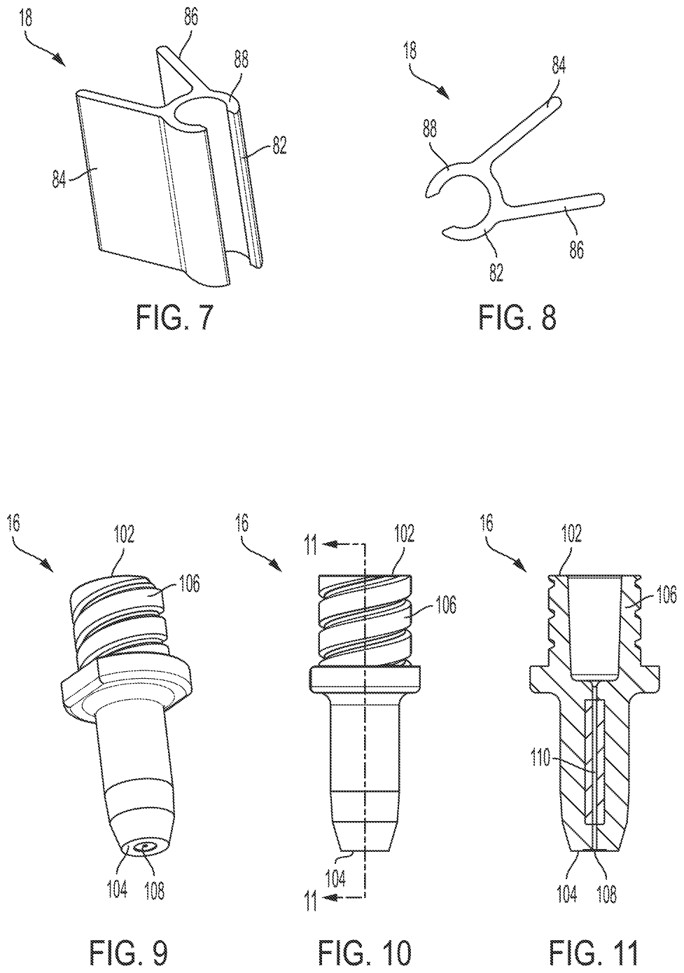

Referring to FIGS. 7 and 8, the dose divider 18 includes a plunger rod attachment portion 82 configured to attach the dose divider 18 to the plunger rod 24 of the syringe 20. The dose divider 18 includes first and second grip portions 84, 86 and a C-shaped body 88, where movement of the grip portions 84, 86 toward each other increase an inner diameter of the C-shaped body 88. Squeezing the first and second grip portions 84, 86 opens up the C-shaped body 88 and allows the C-shaped body 88 to receive the plunger rod 24. After releasing the first and second grip portions 84, 86, the C-shaped body 88 closes and engages the plunger rod 24 to secure the dose divider 18 to the plunger rod 24. A length of the dose divider 18 is configured to allow a predetermined volume of liquid within the syringe 20 to be administered in a first dose with the dose divider 18 subsequently removed to allow a second dose of the predetermined volume of liquid within the syringe 20 to be administered. In other words, with the dose divider 18 secured to the plunger rod 24 and abutting a flange 90 of the syringe barrel 22, the plunger rod 24 can be depressed until a thumb press 92 of the plunger rod 24 engages the dose divider 18. Removal of the dose divider 18 allows the plunger rod 24 to be further depressed until the remaining liquid or desired volume of liquid to be administered.

Referring to FIGS. 9-11, the nozzle spray attachment 16 has a first end 102 and a second end 104 positioned opposite the first end 102, with the nozzle spray attachment 16 having a syringe attachment 106 configured to be secured to the syringe barrel 22 and a nozzle opening 108. The nozzle spray attachment 16 defines a central passageway 110 in fluid communication with the syringe attachment 106 and the nozzle opening 108. The syringe attachment 106 is a luer connector, although other suitable connection arrangements may be utilized. The nozzle spray attachment 16 is configured to be secured to the syringe 20 to administer medicament to a patient via a nasal spray. For instance, the nozzle spray attachment 16 may be similar to and function similarly to the BD Accuspray™ nasal spray system commercially available from Becton, Dickinson and Company.

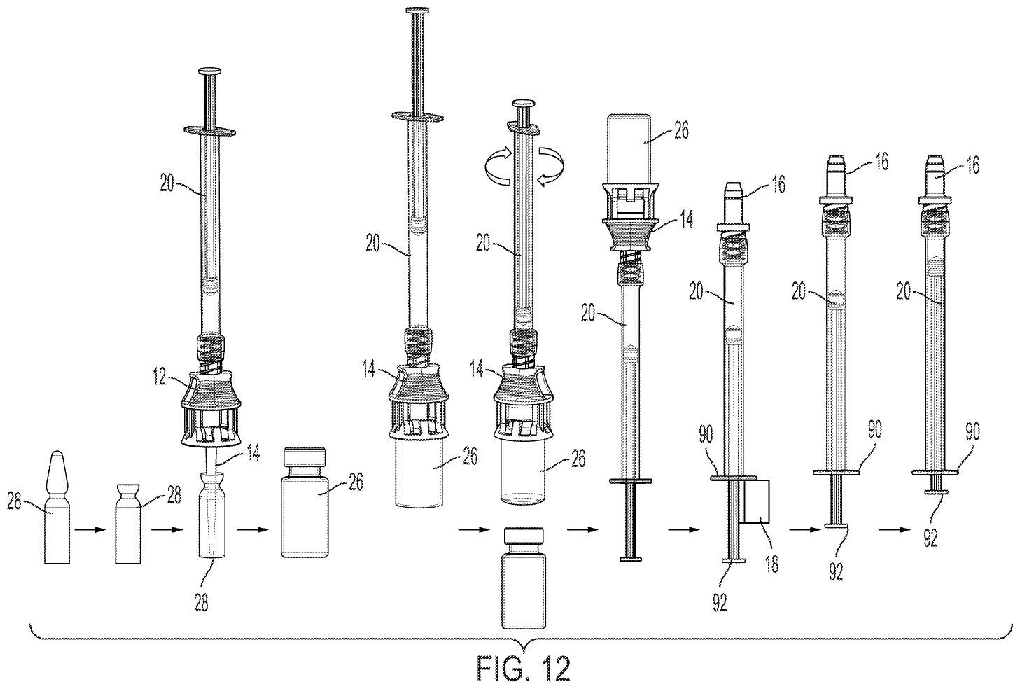

Referring to FIG. 12, a method of using the nasal spray reconstitution system 10, according to one aspect or embodiment, includes: inserting the withdrawal member 14 into the ampoule or other container 28; withdrawing diluent from the container 28 into the syringe 20, which is connected to the vial adapter 12 and the withdrawal member 14; disconnecting the withdrawal member 14 from the vial adapter 12; attaching the vial adapter 12 to the vial 26 containing a lyophilized medicament; transferring the diluent to the vial 26 using the syringe 20 to reconstitute the lyophilized medicament; withdrawing the reconstituted medicament into the syringe 20; removing the vial adapter 12 from the syringe 20; and attaching the nozzle spray attachment 16 to the syringe 20. Reconstituting the lyophilized medicament may include shaking the vial 26 to ensure all of the lyophilized medicament has dissolved. Withdrawing the reconstituted medicament into the syringe 20 may include inverting the vial 26, the vial adapter 12, and the syringe 20. The method may further include attaching the dose divider 18 to the plunger rod 24 of the syringe 20.

The withdrawal member 14 of the system 10 provides for simple and effective extraction of diluent from the ampoule or container 28 with the filter 76 insuring particle-free diluent is withdrawn into the syringe 20. The connection between the vial adapter 12 and the vial 26 provides guidance to ensure a smooth and accurate penetration of the vial 26 by the transfer spike 50 while allowing for a single-handed reconstituting of the medicament. Further, the transfer opening(s) 54 provides for drug vial optimization by minimizing the amount of medicament remaining within the vial 26 after withdrawing liquid from the vial 26 using the vial adapter 12.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

The invention claimed is:

1. A nasal spray reconstitution system comprising:
a vial adapter comprising:
a body having a first end and a second end positioned opposite the first end;
a vial connector configured to secure the body to a vial;
a syringe connector configured to secure the body to a syringe barrel; and
a transfer spike having a pointed end and defining a transfer opening, wherein the body, the syringe connector, and the transfer spike define a passageway in fluid communication with the transfer opening of the transfer spike;
a withdrawal member having a first end and a second end positioned opposite the first end, the withdrawal member defining a withdrawal opening and a central passageway, the withdrawal member secured to the transfer spike via a spike connector, the withdrawal opening of the withdrawal member is in fluid communication with the passageway of the vial adapter; and
a nozzle spray attachment having a first end and a second end positioned opposite the first end, the nozzle spray attachment having a syringe attachment configured to be secured to a syringe barrel and a nozzle opening, the nozzle spray attachment defining a central passageway in fluid communication with the syringe attachment and the nozzle opening.

2. The nasal spray reconstitution system of claim 1, further comprising a syringe having a syringe barrel and a plunger rod, the syringe connector of the vial adapter secured to the syringe barrel.

3. The nasal spray reconstitution system of claim 2, further comprising a vial comprising a lyophilized medicament.

4. The nasal spray reconstitution system of claim 3, further comprising an ampoule comprising a diluent.

5. The nasal spray reconstitution system of claim 1, further comprising a dose divider having a plunger rod attachment portion configured to attach the dose divider to a plunger rod of a syringe.

6. The nasal spray reconstitution system of claim 5, wherein the dose divider comprises first and second grip portions and C-shaped body, and wherein movement of the grip portions toward each other increase an inner diameter of the C-shaped body.

7. The nasal spray reconstitution system of claim 1, wherein the syringe connector comprises a luer connection.

8. The nasal spray reconstitution system of claim 1, wherein the spike connector comprises a luer connection.

9. The nasal spray reconstitution system of claim 1, wherein the withdrawal member tapers in width from the first end of the withdrawal member to the second end of the withdrawal member.

10. The nasal spray reconstitution system of claim 1, further comprising a filter received within the central passageway of the withdrawal member.

11. The nasal spray reconstitution system of claim 1, wherein the body of the vial adapter comprises a grip surface configured to be grasped by a hand of a healthcare provider.

12. The nasal spray reconstitution system of claim 1, wherein the transfer opening of the transfer spike comprises a first opening and a second opening positioned opposite the first opening.

13. The nasal spray reconstitution system of claim 12, wherein the first and second openings of the transfer spike each have a length defined by a direction extending from the first end of the body to the second end of the body and a width defined by a direction extending perpendicular to the length of the first and second openings, the length of the first and second openings is larger than the width of the first and second openings.

14. The nasal spray reconstitution system of claim 13, wherein the length of the first and second openings is at least 5 times larger than the width of the first and second openings.

15. A method of using the nasal spray reconstitution system of claim 1, the method comprising:
inserting the withdrawal member into a container;
withdrawing diluent from the container into a syringe connected to the vial adapter;
disconnecting the withdrawal member from the vial adapter;
attaching the vial adapter to a vial comprising a lyophilized medicament;
transferring the diluent to the vial using the syringe to reconstitute the lyophilized medicament;
withdrawing the reconstituted medicament into the syringe;
removing the vial adapter from the syringe; and
attaching the nozzle spray attachment to the syringe.

16. The method of claim 12, further comprising:
attaching a dose divider to a plunger rod of the syringe.

17. The method of claim 15, wherein the container comprises a glass ampoule.
